(19) United States
(12) Patent Application Publication
(10) Pub. No.: US 2013/0296618 A1
(43) Pub. Date: Nov. 7, 2013

(54) Nano-carbon Antifoulant Materials

(71) Applicant: BAKER HUGHES INCORPORATED, Houston, TX (US)

(72) Inventor: Corina L. Sandu, Pearland, TX (US)

(73) Assignee: Baker Hughes Incorporated, Houston, TX (US)

(21) Appl. No.: 13/871,415

(22) Filed: Apr. 26, 2013

Related U.S. Application Data
(60) Provisional application No. 61/641,107, filed on May 1, 2012.

(51) Int. Cl. C07C 7/20

(52) U.S. Cl. CPC C07C 7/20
USPC 585/3; 585/2

(57) ABSTRACT

A carbon-based additive may be added to a base fluid to form a fluid composition. The fluid composition may inhibit fouling of the base fluid by any fouling-causing components that may be present in the base fluid where the base fluid is an aqueous fluid, a non-aqueous fluid, and combinations thereof. A carbon-based additive may include solid nanoparticles, nanotubes, graphene, graphene oxide, nanoribbons, nanosheets, and combinations thereof. The carbon-based additive may be present in the fluid in an effective amount to inhibit fouling of the fluid by the fouling-causing components, particularly asphaltenes in a non-limiting example.

### RELATED APPLICATIONS

This application claims the benefit of Provisional Patent Application No. 61/641107 filed May 1, 2012, which is incorporated by reference herein in its entirety.

### TECHNICAL FIELD

The present invention relates to a method and a fluid composition having a base fluid and a carbon-based additive in an effective amount to inhibit fouling of the base fluid by any fouling-causing components that may be present within the base fluid, and more particularly relates to inhibiting asphaltene fouling of the base fluid using nano-sized carbon additives.

### BACKGROUND

Asphaltenes are most commonly defined as that portion of crude oil, which is insoluble in heptane. Asphaltenes exist in the form of colloidal dispersions stabilized by other components in the crude oil. They are the most polar fraction of crude oil, and often will precipitate upon pressure, temperature, and compositional changes in the oil resulting from blending or other mechanical or physicochemical processing. Asphaltene precipitation occurs in pipelines, separators, and other equipment. Once deposited, asphaltenes present numerous problems for crude oil producers. For example, asphaltene deposits can plug downhole tubulars, well-bores, choke off pipes and interfere with the functioning of separator equipment.

Many formation fluids, such as petroleum fluids, contain a large number of components with a very complex composition. For the purposes herein, a formation fluid is the product from an oil well from the time it is produced until it is refined. Some of the potentially fouling-causing components present in a formation fluid, for example wax and asphaltenes, are liquid under ambient conditions, but may aggregate or deposit under higher temperatures and pressures. Waxes comprise predominantly high molecular weight paraffinic hydrocarbons, i.e. alkanes. Asphaltenes are typically dark brown to black-colored amorphous solids with complex structures and relatively high molecular weight.

In addition to carbon and hydrogen in the composition, asphaltenes also may contain nitrogen, oxygen and sulfur species. Typical asphaltenes are known to have different solubilities in the formation fluid itself or in certain solvents like carbon disulfide, but are insoluble in solvents like light paraffinics, such as but not including pentane, heptane, etc.

When the formation fluid from a subsurface formation comes into contact with a pipe, a valve, or other production equipment of a wellbore, or when there is a decrease in temperature, pressure, or change of other conditions, asphaltenes may precipitate or separate out of a well stream or the formation fluid while flowing into and through the wellbore to the wellhead. While any asphaltene separation or precipitation is undesirable in and by itself, it is much worse to allow the asphaltene precipitants to accumulate by sticking to the equipment in the wellbore. Any asphaltene precipitants sticking to the wellbore surfaces may narrow pipes; and clog wellbore perforations, various flow valves, and other wellsite and downhole locations. This may result in wellsite equipment failures. It may also slow down, reduce or even totally prevent the flow of formation fluid into the wellbore and/or out of the wellhead.

Similarly, undetected precipitations and accumulations of asphaltenes in a pipeline for transferring crude oil could result in loss of oil flow and/or equipment failure. Crude oil storage facilities could have maintenance or capacity problems if asphaltene precipitations occur. These fluids also carry unstable asphaltenes into the refinery, as well as possibly into finished fuels and products where the asphaltenes cause similar problems for facilities of this nature.

There are large incentives to mitigate fouling in refining. There are large costs associated with shutting down production units because of the fouling components within, as well as the cost to clean the units. The asphaltenes may create an insulating effect within the production unit, reduce the efficiency and/or reactivity, and the like. In either case, reducing the amount of fouling-causing components would reduce the cost of hydrocarbon fluids and the products derived therefrom.

Thus, it would be desirable to develop a method and fluid composition for reducing the amount of fouling-causing components within a base fluid where less of the additive is needed and is more cost effective than current mechanisms being used.

### SUMMARY

There is provided, in one form, a fluid composition having a base fluid, and a carbon-based additive. The base fluid may be an aqueous fluid, a non-aqueous fluid, and combinations thereof. The base fluid may have potentially fouling-causing components, such as asphaltenes in a non-limiting example. The carbon-based additive may be present in the fluid in an effective amount to inhibit fouling of the fluid by the potentially fouling-causing components.

There is further provided in another non-limiting embodiment a method for inhibiting the fouling of a fluid having potentially fouling-causing components. A carbon-based additive may be added or introduced to a base fluid in an effective amount to inhibit fouling of the fluid by the potentially fouling-causing components. The base fluid may include an aqueous fluid, a non-aqueous fluid, and combinations thereof. The carbon-based additive may include solid nanoparticles, nanotubes, graphene, graphene oxide, nanoribbons, nanosheets, and combinations thereof. In another non-limiting embodiment of the method, the carbon-based additive may be or include, but is not limited to a chemically-modified carbon-based additive, a covalently-modified carbon-based additive, a functionalized carbon-based additive, and combinations thereof.

An effective amount of the carbon-based additive in the fluid may prevent or inhibit the agglomeration of any potentially fouling-causing components within the fluid.

### DETAILED DESCRIPTION

It has been discovered that the addition of a carbon-based additive to a base fluid in an effective amount may prevent or inhibit any potentially fouling-causing components from fouling the base fluid. Due to the particular properties of carbon-based materials, such as shape, size, electrical and thermal properties, as well as recent developments in managing the functionalization of the surface of carbon-based nano-materials, it is believed that the ability to either trap or chemically interact with the fouling-causing components is an efficient way to inhibit the fouling of the base fluid by any fouling-causing components. Thus, precipitation of the fouling-causing components is also prevented or inhibited. A carbon-based additive is added to a hydrocarbon stream, so no new elements are introduced into the hydrocarbon stream, i.e. both are made of carbon.

The fouling-causing components may include asphaltenes, solids particles, resins, organic acids, polymers, oxides, sulfides, metals, waxes, and combinations thereof. Prevent or inhibit is defined herein to mean that the carbon-based additive may suppress or reduce the amount of total fouling-causing components within the base fluid if there are actually any fouling-causing components present within the fluid. That is, it is not necessary for fouling to be entirely prevented for the methods and compositions discussed herein to be considered effective, although complete prevention is a desirable goal.

The carbon-based additive may prevent or inhibit fouling of the base fluid by two or more different mechanisms, such as but not limited to a stabilization mechanism, a dispersant mechanism, a radical inhibition mechanism, or combinations thereof. The carbon-based additive may include solid nanoparticles, nanotubes, graphene, graphene oxide, nanoribbons, nanosheets, and combinations thereof. In an alternative embodiment, the carbon-based additive, such as graphene in a non-limiting embodiment, may be modified or altered in a manner to allow the carbon-based additive to utilize the stabilization mechanism and/or the dispersant mechanism. The mechanisms will be described in further detail below. As noted herein, “carbon-based additive” refers to the additive when it has not been chemically modified or functionalized, but the modification or alteration simply enhances the ability of the carbon-based additive.

The stabilization mechanism may occur or be applied when it is desirable for the carbon-based additive, such as the graphene in a non-limiting embodiment, to physically block the ability of the potentially fouling-causing components to agglomerate; thus, the likelihood of precipitation of the asphaltenes is reduced. To physically block the potentially fouling-causing components from agglomerating, it may be desirable for the graphene to be layered in a non-limiting embodiment. The graphene may have a first graphene sheet bonded to at least a second graphene sheet to form a layered graphene. The layered graphene may have up to about 6 layers of graphene, or alternatively from about 2 graphene sheets independently to about 4 layers of graphene. Either a single sheet of graphene or the layered graphene is capable of physically blocking the potentially fouling-causing components (e.g. asphaltene) from polymerizing. A covalent bond may hold two graphene sheets together; more than two graphene sheets may be covalently bonded together where a covalent bond is formed between the first graphene sheet to the second graphene sheet, between the second graphene sheet and a third graphene sheet, and so on.

The interlayer distance between each layer within the layered graphene may be about 0.01 nm up to about 1 nm, or alternatively from 0.01 nm independently to about 0.35 nm. Chemical functionalization or functional groups may be intercalated between the graphene layers to further inhibit fouling of the fluids, such as alkyl amines in one non-limiting example. The layering of graphene may allow for 2-D and/or 3-D structures where the 3-D structures may be a type of covalent organic framework (COF). COFs have a crystalline extended organic structure having strong covalent bonds therein. One non-limiting embodiment of the layered graphene as a COF may have at least one dimension ranging from about 2.7 nm to about 3 nm.

The stabilization mechanism may be performed in a base fluid at a temperature ranging from about ambient independently to about 1000C, or alternatively from about 200C independently to about 800C once the carbon-based additive has been added to the base fluid. The effective amount of the carbon-based additive added to the base fluid for the stabilization effect to occur may range from about 1 ppm independently to about 10000 ppm, or alternatively from about 5 ppm independently to about 2000 ppm, or 5 ppm independently to about 500 ppm. “Independently” is defined herein to mean that any lower threshold may be used together with any upper threshold to give a suitable alternative range. An effective amount is defined herein as an amount added to inhibit or prevent the potentially fouling-causing components from agglomerating together.

The dispersant mechanism may occur or be applied when the carbon-based additive is functionalized, chemically-modified, covalently-modified, and combinations thereof. The modification of the carbon-based additive depends on the desired functionality of the carbon-based additive. The modified or altered carbon-based additive may then prevent or inhibit potentially fouling-causing components from agglomerating or polymerizing or the forming of free radicals. The modification and/or functionalization of the carbon-based additive may improve the antifouling effect of the carbon-based additive as compared with an otherwise identical carbon-based additive that has not been modified or functionalized. The carbon-based additive may be a layered graphene that may be modified or functionalized in a non-limiting example.

The functionalized carbon-based additive may have at least one functional group in a non-limiting embodiment. The functional group may include, but not necessarily be limited to, a sulfonate, a sulfate, a sulfosuccinate, a thiosulfate, a succinate, a carboxylate, a hydroxyl, a glucoside, an ethoxylate, a propoxylate, a phosphate, an ethoxylate, an ether, an amine, an amide, a carboxylic acid, an epoxide ring opening, an oligothiophine, a protoporphyrin, polystyrene, an epoxy, a carbonyl, a carboxyl, a diazonium ion, and combinations thereof. In another non-limiting embodiment, the carbon-based additive may have at least one functional group that is a polar group, a non-polar group, and combinations thereof.

In an alternative embodiment, the chemically-modified carbon-based additive may have at least one functional group that may include, but is not necessarily limited to, SH, NH₂, NHCO, OH, COON, F, Br, Cl, I, H, R—NH, R—O, R—S, CO, COCl, SOCl, and combinations thereof; where R may be or include at least one low molecular weight organic chains with a carbon number greater than 5.

In another non-limiting embodiment, the covalently-modified carbon-based additive may have at least one covalent modification that may include, but is not necessarily limited to, oxidation; free radical additions, such as addition of carbenes, nitrenes and other radicals; arylamine attachment via diazonium chemistry; and combinations thereof.

These modifications and/or functionalizations to the carbon-based additive, such as graphene in a non-limiting example, allow the dispersant effect to occur because the modified/functionalized carbon-based additive may bind to a potentially fouling-causing component (e.g. asphaltene), which chemically inhibits the potentially fouling-causing component from binding or polymerizing to another potentially fouling-causing component. In such an embodiment, a single graphene sheet may have functional groups attached to the graphene sheet.

The dispersant mechanism or effect of preventing or inhibiting the potentially fouling-causing components from agglomerating by functionalizing or modifying the carbon-based additive may be performed at a temperature ranging from about ambient independently to about 1000C, or alternatively from about 200C independently to about 800C, once the carbon-based additive has been added to the base fluid. While the dispersant mechanism functions in the same temperature ranges as the stabilization mechanism, the temperatures for dispersant mechanism of inhibiting or preventing the potentially fouling-causing components from agglomerating may function better at lower temperatures within the range than the stabilization mechanism. There is less agglomeration of the potentially fouling-causing components at lower temperatures, so it is easier to disperse the carbon-based additive into the base fluid.

The effective amount of the carbon-based additive added to the base fluid may range from about 1 ppm independently to about 10000 ppm, or alternatively from about 5 ppm independently to about 2000 ppm, or 5 ppm independently to about 500 ppm.

Each component of the carbon-based additive may have at least one dimension less than about 999 nm, or alternatively less than about 500 nm. In an alternative embodiment, the average particle size may be less than about 999 nm, or less than 500 nm in another non-limiting embodiment. As defined herein, the term ‘average particle size’ means that some particles may be larger than a designated amount, such as 999 nm in the aforementioned example, or that some particles may be smaller than the designated amount, but that the average is within the range given.

In one non-limiting embodiment, a plurality of inorganic nanoparticles may be optionally added to the base fluid in addition to the carbon-based additive. The inorganic nanoparticles may include metal oxides, amorphous materials, and combinations thereof. The metal oxides may include silica oxide, magnesium oxide, iron oxide, manganese oxide, copper oxide, zinc oxide, and combinations thereof. The amorphous materials may include, but are not limited to clays, crystalline structures, combinations thereof, and the like.

The base fluid may be an aqueous fluid, a non-aqueous fluid, and combinations thereof. In a non-limiting embodiment, the base fluid may be a producing fluid, a formation fluid, a well stream, a process refinery stream, a finished fuel, and combinations thereof. A surfactant may be added to the base fluid in an amount effective to suspend the carbon-based additive in the base fluid. Surfactants are generally considered optional, but may be used to improve the quality of the dispersion of the carbon-based additive within the base fluid.

Expected suitable surfactants may include, but are not necessarily limited to non-ionic, anionic, cationic, amphoteric surfactants and zwitterionic surfactants, janus surfactants, and blends thereof. Suitable nonionic surfactants may include, but are not necessarily limited to, alkyl polyglycosides, sorbitan esters, methyl glucoside esters, amine ethoxylates, diamine ethoxylates, polyglycerol esters, alkyl ethoxylates, alcohols that have been polypropoxylated and/or polyethoxylated or both. Suitable anionic surfactants may include alkali metal alkyl sulfates, alkyl ether sulfonates, alkyl sulfonates, alkyl aryl sulfonates, linear and branched alkyl ether sulfates and sulfonates, alcohol polypropoxylated sulfates, alcohol polyethoxylated sulfates, alcohol polypropoxylated polyethoxylated sulfates, alkyl disulfonates, alkylaryl disulfonates, alkyl disulfates, alkyl sulfosuccinates, alkyl ether sulfates, linear and branched ether sulfates, alkali metal carboxylates, fatty acid carboxylates, and phosphate esters. Suitable cationic surfactants may include, but are not necessarily limited to, arginine methyl esters, alkanolamines and alkylenediamides. Suitable surfactants may also include surfactants containing a non-ionic spacer-arm central extension and an ionic or nonionic polar group. Other suitable surfactants may be dimeric or gemini surfactants, cleavable surfactants, janus surfactants and extended surfactants, also called extended chain surfactants.

In the foregoing specification, the invention has been described with reference to specific embodiments thereof, and has been described as effective in providing methods and compositions for inhibiting and preventing fouling-causing components in fluids, such as asphaltenes, from fouling the fluids. However, it will be evident that various modifications and changes can be made thereto without departing from the broader spirit or scope of the invention as set forth in the appended claims. Accordingly, the specification is to be regarded in an illustrative rather than a restrictive sense. For example, specific fluids, carbon-based additives, and fouling-causing components falling within the claimed parameters, but not specifically identified or tried in a particular composition or method, are expected to be within the scope of this invention.

The present invention may suitably comprise, consist or consist essentially of the elements disclosed and may be practiced in the absence of an element not disclosed. For instance, the fluid composition and the method may consist of or consist essentially of a carbon-based additive added to a base fluid in an effective amount to inhibit or prevent fouling of the base fluid by the fouling-causing components as compared to the base fluid absent any carbon-based additive.

The words “comprising” and “comprises” as used throughout the claims, are to be interpreted to mean “including but not limited to” and “includes but not limited to”, respectively.

What is claimed is:

1. A fluid composition comprising:
a base fluid having potentially fouling-causing components, wherein the base fluid is selected from the group consisting of an aqueous fluid, a non-aqueous fluid, and combinations thereof; and
a carbon-based additive having carbon components, wherein at least one carbon component is selected from the group consisting of solid nanoparticles, nanotubes, graphene, graphene oxide, nanoribbons, nanosheets, and combinations thereof; and
wherein the carbon-based additive is present in the base fluid in an effective amount to inhibit fouling of the base fluid by the fouling-causing components as compared to the base fluid absent any carbon-based additive.

2. The fluid composition of claim 1, wherein the fouling-causing components are selected from the group consisting of asphaltenes, resins, organic acids, polymers, oxides, sulfides, metals, waxes, and combinations thereof.

3. The fluid composition of claim 1, wherein each carbon component has at least one dimension less than about 999 nm.

4. The fluid composition of claim 1, wherein the effective amount of the carbon-based additive in the base fluid ranges from about 1 ppm to about 10000 ppm.

5. The fluid composition of claim 1, wherein the graphene comprises a first graphene sheet bonded to at least a second graphene sheet to form a layered graphene.

6. The fluid composition of claim 1 further comprising metal oxide nanoparticles selected from the group consisting of silica oxide, magnesium oxide, iron oxide, manganese oxide, copper oxide, zinc oxide, and combinations thereof.

7. The fluid composition of claim 1 further comprising a surfactant in an amount effective to suspend the carbon-based additive in the base fluid.

8. The fluid composition of claim 1, wherein the carbon-based additive is selected from the group consisting of a chemically-modified carbon-based additive, a covalently-modified carbon-based additive, a functionalized carbon-based additive, and combinations thereof; wherein the modification and/or functionalization of the carbon-based additive improves the antifouling effect of the carbon-based additive as compared with an otherwise identical carbon-based additive that has not been modified or functionalized.

9. The fluid composition of claim 8, wherein the functionalized carbon-based additive comprises at least one functional group selected from the group consisting of a sulfonate, a sulfate, a sulfosuccinate, a thiosulfate, a succinate, a carboxylate, a hydroxyl, a glucoside, an ethoxylate, a propoxylate, a phosphate, an ethoxylate, an ether, an amine, an amide, a carboxylic acid, an epoxide ring opening, an oligothiophine, a protoporphyrin, polystyrene, an epoxy, a carbonyl, a carboxyl, a diazonium ion, and combinations thereof; wherein the chemically-modified carbon-based additive has at least one functional group selected from the group consisting of SH, NH₂, NHCO, OH, COON, F, Br, Cl, I, H, R—NH, R—O, R—S, CO, COCl, SOCl, and combinations thereof where R is selected from the group consisting of low molecular weight organic chains with a carbon number greater than 5; and wherein the covalently-modified additive comprises at least one covalent modification selected from the group consisting of oxidation; free radical additions; arylamine attachment via diazonium chemistry; and combinations thereof.

10. A fluid composition comprising:
a base fluid having potentially fouling-causing components are selected from the group consisting of asphaltenes, resins, organic acids, polymers, oxides, sulfides, metals, waxes, and combinations thereof, wherein the base fluid is selected from the group consisting of an aqueous fluid, a non-aqueous fluid, and combinations thereof; and
a carbon-based additive comprising carbon components, wherein at least one carbon component is selected from the group consisting of a carbon-based additive selected from the group consisting of solid nanoparticles, nanotubes, graphene, graphene oxide, nanoribbons, nanosheets, and combinations thereof; wherein each component has at least one dimension less than about 999 nm; and
wherein the carbon-based additive is present in the base fluid in an effective amount to inhibit fouling of the base fluid by the components as compared to the base fluid absent any carbon-based additive.

11. A method for inhibiting the fouling of a base fluid having potentially fouling-causing components, wherein the method comprises adding a carbon-based additive to a base fluid; wherein the carbon-based additive comprises at least one carbon component selected from the group consisting of solid nanoparticles, nanotubes, graphene, graphene oxide, nanoribbons, nanosheets, and combinations thereof in an effective amount to inhibit fouling of the fluid by the potentially fouling-causing components as compared to the base fluid absent any carbon-based additive; wherein the base fluid is selected from the group consisting of an aqueous fluid, a non-aqueous fluid, and combinations thereof.

12. The method of claim 11, wherein the fouling-causing components are selected from the group consisting of asphaltenes, resins, organic acids, polymers, oxides, sulfides, metals, waxes, and combinations thereof.

13. The method of claim 11 further comprising inhibiting fouling by a method selected from the group consisting of physically blocking the ability of the fouling-causing components to agglomerate, depolymerizing the fouling-causing components, and a combination thereof.

14. The method of claim 11, wherein each carbon component has at least one dimension less than about 999 nm.

15. The method of claim 11, wherein the effective amount of the carbon-based additive ranges from about 1 ppm to about 10000 ppm.

16. The method of claim 11, wherein the graphene comprises a first graphene sheet bonded to at least a second graphene sheet to form a layered graphene.

17. The method of claim 11, further comprising metal oxide nanoparticles selected from the group consisting of silica oxide, magnesium oxide, iron oxide, manganese oxide, copper oxide, zinc oxide, and combinations thereof.

18. The method of claim 11, wherein the carbon-based additive is selected from the group consisting of a chemically-modified carbon-based additive, a covalently-modified carbon-based additive, a functionalized carbon-based additive, and combinations thereof; wherein the modification and/or functionalization of the carbon-based additive improves the antifouling effect of the additive as compared with an otherwise identical carbon-based additive that has not been modified or functionalized.

19. The method of claim 18, wherein the functionalized carbon-based additive comprises at least one functional group selected from the group consisting of a sulfonate, a sulfate, a sulfosuccinate, a thiosulfate, a succinate, a carboxylate, a hydroxyl, a glucoside, an ethoxylate, a propoxylate, a phosphate, an ethoxylate, an ether, an amine, an amide, a carboxylic acid, an epoxide ring opening, an oligothiophine, a protoporphyrin, polystyrene, an epoxy, a carbonyl, a carboxyl, a diazonium ion, and combinations thereof; wherein the chemically-modified carbon-based additive has at least one functional group selected from the group consisting of SH, NH₂, NHCO, OH, COON, F, Br, Cl, I, H, R—NH, R—O, R—S, CO, COCl, SOCl, and combinations thereof where R is selected from the group consisting of low molecular weight organic chains with a carbon greater than 5; and wherein the covalently-modified additive comprises at least one covalent modification selected from the group consisting of oxidation; free radical additions; arylamine attachment via diazonium chemistry; and combinations thereof.

20. A method for inhibiting the fouling of a base fluid having potentially fouling-causing components, the method comprising:
adding a carbon-based additive to a base fluid, wherein the carbon-based additive comprises at least one carbon component having at least one dimension less than about 999 nm; and wherein at least one carbon component is selected from the group consisting of a carbon-based additive selected from the group consisting of solid nanoparticles, nanotubes, graphene, graphene oxide, nanoribbons, nanosheets, and combinations thereof in an effective amount to inhibit fouling of the fluid by the potentially fouling-causing components as compared to the base fluid absent any carbon-based additive; wherein the base fluid is selected from the group consisting of an aqueous fluid, a non-aqueous fluid and combinations thereof; and
inhibiting the fouling of the base fluid by any potentially-fouling causing components within the fluid; wherein the fouling-causing components are selected from the group consisting of asphaltenes, resins, organic acids, polymers, oxides, sulfides, metals, and combinations thereof.
